(19) United States
(12) Patent Application Publication
(10) Pub. No.: US 2010/0008893 A1
(43) Pub. Date: Jan. 14, 2010

(54) BACILLUS VELEZENSIS STRAIN

(75) Inventor: Simon Rucker, Rocky Mount, VA (US)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/500,859

(22) Filed: Jul. 10, 2009

Related U.S. Application Data
(60) Provisional application No. 61/079,926, filed on Jul. 11, 2008.

(51) Int. Cl. A01N 63/02; C12N 1/20; A01P 1/00

(52) U.S. Cl. 424/93.46; 435/252.5

(57) ABSTRACT

The present invention relates to Bacillus velezensis strain NRRL B 50150 and methods and compositions for preventing and/or reducing biofilm formation on surfaces and/or planktonic proliferation in aqueous environments, especially in domestic/household and industrial settings. The present invention also relates to deodorizing liquid compositions which are designed to be applied in the areas of pet care, toilet care, carpet care, and garbage collections or processes, management of industrial wastes, including sludge processing, landfill and composting, and odor control of livestock production processes and other organic wastes.

### CROSS-REFERENCE TO RELATED APPLICATIONS

This application claims priority or the benefit under 35 U.S.C. 119 of U.S. provisional application No. 60/079,926 filed Jul. 11, 2008, the contents of which are fully incorporated herein by reference.

### CROSS-REFERENCE TO DEPOSITED MICROORGANISMS

The present application refers to deposited microorganisms. The contents of the deposited microorganisms are fully incorporated herein by reference.

### FIELD OF THE INVENTION

The present invention relates to Bacillus velezensis strain NRRL B-50150 and methods and compositions for preventing and/or reducing biofilm formation on surfaces and/or planktonic proliferation in aqueous environments, especially in domestic/household and industrial settings. The present invention also relates to deodorizing liquid compositions which are designed to be applied in the areas of pet care, toilet care, carpet care, and garbage collections or processes, management of industrial wastes, including sludge processing, landfill and composting, and odor control of livestock production processes and other organic wastes.

### BACKGROUND OF THE INVENTION

Biofilm formation and planktonic proliferation by undesired microorganisms are well known phenomena in domestic as well as industrial settings. For instance, toilet bowls harbor undesirable bacteria on surfaces and in solution that can contribute to a noticeably fouled appearance of the bowl. Further, the presence of undesired microorganisms in the bowl may cause dispersion of aerosols when flushing. Massive biofilm formation and planktonic proliferation in water systems, e.g., pipes, pumps and vessels, are known to cause health care risks, corrosion, and aesthetic problems.

Preventing or reducing biofilm formation and/or planktonic proliferation by undesirable microorganisms traditionally requires the use of dispersants, surfactants, enzymes, microbes, antimicrobial agents, biocides, boil-out procedures, and/or chemicals.

U.S. Pat. No. 5,171,591 concerns controlling or eliminating undesired bacteria in or on certain food or food contact surfaces using parasitic bacteria of the genus Bdellovibrio.

U.S. Pat. No. 5,242,593 concerns a method for reducing the buildup of slime and/or film in water circulation systems by adding non-sessile microbes in single form to the circulating water.

U.S. Pat. No. 5,360,517 discloses a process of regulating the growth of the microbial/bacterial flora existing in an aqueous papermaking circuit/process stream comprising introducing an effective disinfectant amount of bacteria of the species Staphylococcus carnosus.

U.S. Pat. No. 5,863,882 concerns liquid cleaning and sanitizing formulations comprising a sanitizing composition, viable Bacillus spores, and surfactants capable of reducing four pathogenic microorganisms.

AU Patent No. 719544 concerns a method of controlling the number of pathogenic bacteria in a body of water by adding non-pathogenic gram positive bacteria.

WO 2006/031554 disclose a method of preventing, removing, reducing or disrupting biofilms on surfaces by contacting said surface with an alpha-amylase derived from a bacterium.

Bacillus velezensis strain SB3190 is able to produce amylase and functions as an effective biological control agent. Bacillus velezensis strain SB3190 is currently included in a product marketed by Novozymes for a different application.

Though methods of reducing and preventing biofilm formation and planktonic proliferation of undesired microorganisms are known in the art there is still a need for methods and compositions for doing so.

### SUMMARY OF THE INVENTION

The present invention relates to a biologically pure culture of Bacillus velezensis strain NRRL B-50150. Bacillus velezensis strain NRRL B-50150 is a bacteriophage-resistant (phage-resistant) variant of Bacillus velezensis strain SB3190. In order to propagate Bacillus velezensis strain NRRL B-50150 to a number large enough to allow broad application of this strain, repeated, large-scale fermentation is required. It is known that the natural introduction of native bacteriophage can occur in standard large-scale fermentation systems over repeated growth events or batches. Such an infection can rapidly lead to a complete loss of the culture within hours or days, negating the ability to provide the strain for practical applications. Bacillus velezensis strain NRRL B-50150 is resistant to such a phage, and therefore maintains growth and realizes the benefits described herein.

The present invention also relates to methods and compositions for reducing and/or preventing biofilm formation and/or planktonic proliferation in aqueous environments.

Bacillus velezensis strain NRRL B-50150 is able to produce amylase, which catalyzes the degradation of the principal chemical components of drain residues, such as starches.

This invention also relates to a liquid deodorizing composition comprising Bacillus velezensis strain NRRL B-50150 in an aqueous solution, e.g., distilled water, tap water, a saline solution or other aqueous solution.

The present invention is also directed to a drain opener formulation comprising Bacillus velezensis strain NRRL B-50150.

The present invention also relates to a sanitizing composition comprising Bacillus velezensis strain NRRL B-50150 in an aqueous solution.

### DETAILED DESCRIPTION OF THE INVENTION

### Culture

The present invention is directed to a biologically pure culture of Bacillus velezensis strain NRRL B-50150.

Methods for Preventing and/or Reducing Biofilm Formation

The invention also relates to methods for preventing and/or reducing biofilm formation on a surface comprising subjecting said surface to Bacillus velezensis strain NRRL B-50150.

The term “biofilm formation” means the formation of a slime layer or film by undesired microorganisms on a surface. Biofilm formation is a consequence of growth of undesired microorganisms which attach singly or in colonies to a surface.

The term “surface” refers to any surface, preferably hard surfaces, which may be prone to biofilm formation and adhesion of microorganisms. Examples of contemplated surfaces include hard surfaces made from one or more of the following materials: metal, plastic, rubber, board, glass, wood, paper, concrete, rock, marble, gypsum and ceramic materials, such as porcelain, which optionally are coated, for example, with paint or enamel. Examples of soft surfaces include surfaces made of fibers of any kind (e.g., yarns, textiles, vegetable fibers, rock wool, and hair); or any porous surface; skin (human or animal); keratinous materials (e.g., nails); and internal organs (e.g., lungs).

Hard surfaces are, for instance, found in bathrooms, e.g., fixtures, sinks, bathtubs, toilet bowls, and rinse water reservoirs; in cooling towers; water treatment plants; water tanks; dairy, food processing plants etc.; chemical or pharmaceutical process plants; or medical devices (e.g., catheters, orthopedic devices, and implants). Biofilm prone surfaces may also be porous surfaces. Porous surfaces can, for instance, be present in filters, e.g., membrane filters.

Methods for Preventing and/or Reducing Planktonic Proliferation

The invention also relates to methods for preventing and/or reducing planktonic proliferation of microorganism(s), comprising subjecting said microorganism(s) in aqueous solution to Bacillus velezensis strain NRRL B-50150.

The term “planktonic proliferation” means growth of undesired microorganisms, preferably undesired bacteria, in an aqueous environment, such as a body of water. The undesired microorganisms typically occur freely in the aqueous environment. Examples of contemplated aqueous environments are rinse water in toilet bowls and cooling water circulated in plants.

The composition may comprise other active and/or inactive ingredients.

The terms “effective amount”, “effective concentration” or “effective dosage” are defined herein as the amount, concentration or dosage of one or more bacteria strains that can reduce and/or prevent biofilm formation caused by undesired microorganisms on a surface and/or reduce and/or prevent planktonic proliferation of undesired microorganisms in an aqueous environment. The actual effective dosage in absolute numbers depends on factors including: the undesired microorganism(s) in question; whether the aim is prevention or reduction; the contact time between the strain(s) or composition comprising said strain(s); other ingredients present, and also the surface or aqueous environment in question. An effective dosage of Bacillus velezensis strain NRRL B-50150 is in the range from 1 to 1×10⁸ cfu/ml, preferably 50 to 1×10⁷ cfu/ml. Further, in an embodiment the ratio between the Bacillus velezensis strain NRRL B 50150 and the undesired microorganism(s) in question may be between 1:100,000 and 100,000:1 (bacterial strain:undesired microorganism), preferably 1:10,000 to 10,000:1, more preferably 1:1,000 to 1,000:1, more preferably 1:100 to 100:1, even more preferably 1:10 to 10:1.

In general, environments that receive high loads of undesirable microorganisms and nutrients require high doses of mitigating bacteria strains, while environments with low loads of undesirable organisms require lower doses of mitigating bacteria strains. Further, for instance, preventing biofilm formation on surfaces or preventing planktonic formation in aqueous environments, in general, require lower doses of Bacillus velezensis strain NRRL B-50150 than reducing biofilm formation on corresponding surfaces or reducing the number of already existing undesired microorganism(s) in corresponding aqueous environments.

Consequently, a method of the invention can be used for inhibiting growth (i.e., leading to reduced biofilm formation) of one or more undesired microorganisms, preferably bacteria already present on a surface or already present in an aqueous environment. In another embodiment the invention relates to preventing and/or significantly retarding biofilm formation on an essentially clean surface (i.e., surface with essentially no undesired microorganisms) and/or planktonic proliferation in essentially clean water (i.e., aqueous environment containing essentially no undesired microorganisms). In other words, Bacillus velezensis strain NRRL B-50150 protects the surface and/or aqueous environment against future growth of one or more undesired microorganisms. A method of the invention may result in reduction or even elimination/removal of already existing undesired microorganisms. Bacillus velezensis strain NRRL B-50150 may in a preferred embodiment be applied to the surface in question and/or or added to the aqueous environment in question periodically. Periodically means that the method of the invention may be reiterated or repeated over a period of time, e.g., every minute, hour, day, week, month, etc. As mentioned above, the effect may not last for a long period of time. It may require redosing of Bacillus velezensis strain NRRL B-50150. For instance, when the surface and aqueous environment is on the inside of a toilet bowl and the rinsing water in the toilet bowl, respectively, redosing may take place (periodically), e.g., with every flushing. Bacillus velezensis strain NRRL B-50150 may, for instance, be incorporated into a rim block.

A method of the invention may also be carried out by manually and/or mechanically subjecting (i.e., applying or contacting) Bacillus velezensis strain NRRL B-50150 or a composition comprising Bacillus velezensis strain NRRL B-50150 to the surface in question.

### Undesired Microorganisms

In context of the invention the term “undesired microorganisms” means microorganisms that may result in an effect considered to be negative on the surface in question and/or in the aqueous environment in question, especially in domestic or industrial settings. Examples of such negative effects include odor, corrosion, pitting, or other degradation of material; infection; staining or otherwise making a surface appear aesthetically unpleasing. Undesired microorganisms also include pathogenic microorganisms, especially pathogenic bacteria.

By using Bacillus velezensis strain NRRL B-50150 in an effective amount biofilm formation on surfaces and/or planktonic proliferation in aqueous environments can be reduced and/or prevented.

In a preferred embodiment the surface in question prone to biofilm formation may be subjected to Bacillus velezensis strain NRRL B-50150 as a preventative measure prior to any biofilm formation/buildup. This results in that significantly less biofilm is formed. Alternatively, if a biofilm has already formed, or at the first sign of biofim buildup a method of the invention may be used to reduce further biofilm formation. A method of the invention may even result in partly or complete removal of the biofilm.

Examples of undesired microorganisms include those disclosed below.

Undesired microorganisms include, but are not limited to, aerobic bacteria or anaerobic bacteria, Gram positive and Gram negative, fungi (yeast or filamentous fungus), algae, and/or protozoa. Contemplated bacteria include bacteria selected from the group consisting of. Pseudomonas spp. including Pseudomonas aeruginosa, Azotobacter vinelandii, Escherichia coli, Corynebacterium diphteriae, Clostridium botulinum, Streptococcus spp., Acetobacter, Leuconostoc, Betabacterium, Pneumococcus, Mycobacterium tuberculosis, Aeromonas, Burkholderia, Flavobacterium, Salmonella, Staphylococcus, Vibrio spp., Listeria spp., and Legionella spp.

In a preferred embodiment, the undesired microorganism is an aerobic bacterium. In a more preferred embodiment, the aerobic bacterium is an Aeromonas strain. In another more preferred embodiment, the aerobic bacterium is a Burkholderia strain. In another more preferred embodiment, the aerobic bacterium is a Flavobacterium strain. In another more preferred embodiment, the aerobic bacterium is a Microbacterium strain. In another more preferred embodiment, the aerobic bacterium is a Pseudomonas strain. In another more preferred embodiment, the aerobic bacterium is a Salmonella strain. In another more preferred embodiment, the aerobic bacterium is a Staphylococcus strain. In another more preferred embodiment, the aerobic bacterium is from the family Enterobacteriaceae (including e.g., Escherichia coli).

In a most preferred embodiment, the aerobic bacterium is Burkholderia cepacia. In another most preferred embodiment, the aerobic bacterium is a Microbacterium imperials or Mycobacterium tuberculosis. In another most preferred embodiment, the aerobic bacterium is Pseudomonas aeruginosa. In another most preferred embodiment, the aerobic bacterium is Pseudomonas fluorescens. In another most preferred embodiment, the aerobic bacterium is Pseudomonas oleovorans. In another most preferred embodiment, the aerobic bacterium is Pseudomonas pseudoalcaligenes. In another most preferred embodiment, the aerobic bacterium is Salmonella enteritidis. In another most preferred embodiment, the aerobic bacterium is Staphylococcus aureus. In another most preferred embodiment, the aerobic bacterium is Staphylococcus epidermidis.

In another most preferred embodiment the bacterium is Listeria monocytogenes.

In another most preferred embodiment the bacteria is Legionella adelaidensis. In another most preferred embodiment the bacteria is Legionella pneumophila. In another most preferred embodiment the bacteria is Legionella feeleii. In another most preferred embodiment the bacteria is Legionella moravica.

In another embodiment the bacteria is Vibrio harveyi, Vibrio fischerli, and/or Vibrio alginolyticus.

In another preferred embodiment, the microorganism is an anaerobic bacterium. In another more preferred embodiment, the anaerobic bacterium is a Desulfovibrio strain. In another most preferred embodiment, the anaerobic bacterium is Desulfovibrio desulfuricans.

In another preferred embodiment, the undesired microorganism is a fungus such as a yeast or filamentous fungus. In another more preferred embodiment, the yeast is a Candida strain. In another most preferred embodiment, the yeast is Candida albicans.

### Composition of the Invention

The invention also relates to a composition comprising Bacillus velezensis strain NRRL B-50150. In an embodiment the composition further comprises a surfactant or one or more other ingredients mentioned below.

### Surfactants

The surfactants may be non-ionic including semi-polar and/or anionic and/or cationic and/or zwitterionic. The surfactant(s) should cause as little harm to the bacteria culture's activity as possible.

The surfactants may be present in the composition at a level of from 0.01% to 60% by weight.

When included therein the composition usually contains from about 0 to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

When included therein the composition usually contains from about 0 to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

### Other Ingredients

The composition may comprise one or more enzymes. Examples of contemplated enzymes are mentioned in the “Enzymes”-section.

Other ingredients include, but are not limited to, dispersants, stabilizers, anti-microbial agents, fragrances, dyes, and biocides.

### Enzymes

One or more enzymes may be present in a composition of the invention. Especially contemplated enzymes include proteases, alpha-amylases, cellulases, lipases, peroxidases/oxidases, pectate lyases, and mannanases, or mixtures thereof.

Proteases: Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274. Preferred commercially available protease enzymes include ALCALASE™, SAVINASE™, PRIMASE™, DURALASE™, DYRAZYM™, ESPERASE™, EVERLASE™, POLARZYME™ and KANNASE™, LIQUANASE™ (Novozymes A/S), MAXATASE™, MAXACAL™, MAXAPEM™, PROPERASE™, PURAFECT™, PURAFECT OxP™, FN2™, and FN3™ (Genencor International Inc.).

Lipases: Suitable lipases include those of bacterial or fungal origin. Chemically modified or protein engineered mutants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include LIPOLASE™ and LIPOLASE ULTRA™, LIPOZYME™, and LIPEX™ (Novozymes A/S).

Cutinase: The method of the invention may be carried out in the presence of cutinase classified in EC 3.1.1.74.

The cutinase used according to the invention may be of any origin. Preferably cutinases are of microbial origin, in particular of bacterial, of fungal or of yeast origin.

Cutinases are enzymes which are able to degrade cutin. In a preferred embodiment, the cutinase is derived from a strain of Aspergillus, in particular Aspergillus oryzae, a strain of Alternaria, in particular Alternaria brassiciola, a strain of Fusarium, in particular Fusarium solani, Fusarium solani pisi, Fusarium roseum culmorum, or Fusarium roseum sambucium, a strain of Helminthosporum, in particular Helminthosporum sativum, a strain of Humicola, in particular Humicola insolens, a strain of Pseudomonas, in particular Pseudomonas mendocina, or Pseudomonas putida, a strain of Rhizoctonia, in particular Rhizoctonia solani, a strain of Streptomyces, in particular Streptomyces scabies, or a strain of Ulocladium, in particular Ulocladium consortiale. In a most preferred embodiment the cutinase is derived from a strain of Humicola insolens, in particular the strain Humicola insolens DSM 1800. Humicola insolens cutinase is described in WO 96/13580 which is herby incorporated by reference. The cutinase may be a variant, such as one of the variants disclosed in WO 00/34450 and WO 01/92502, which are hereby incorporated by reference. Preferred cutinase variants include variants listed in Example 2 of WO 01/92502, which is hereby specifically incorporated by reference.

Preferred commercial cutinases include NOVOZYM™ 51032 (available from Novozymes A/S, Denmark).

The method of the invention may be carried out in the presence of phospholipase classified as EC 3.1.1.4 and/or EC 3.1.1.32. As used herein, the term phospholipase is an enzyme which has activity towards phospholipids. Phospholipids, such as lecithin or phosphatidylcholine, consist of glycerol esterified with two fatty acids in an outer (sn-1) and the middle (sn-2) positions and esterified with phosphoric acid in the third position; the phosphoric acid, in turn, may be esterified to an amino-alcohol. Phospholipases are enzymes which participate in the hydrolysis of phospholipids. Several types of phospholipase activity can be distinguished, including phospholipases A₁ and A₂ which hydrolyze one fatty acyl group (in the sn-1 and sn-2 position, respectively) to form lysophospholipid; and lysophospholipase (or phospholipase B) which can hydrolyze the remaining fatty acyl group in lysophospholipid. Phospholipase C and phospholipase D (phosphodiesterases) release diacyl glycerol or phosphatidic acid respectively.

The term phospholipase includes enzymes with phospholipase activity, e.g., phospholipase A (A₁ or A₂), phospholipase B activity, phospholipase C activity or phospholipase D activity. The term “phospholipase A” used herein in connection with an enzyme of the invention is intended to cover an enzyme with Phospholipase A₁ and/or Phospholipase A₂ activity. The phospholipase activity may be provided by enzymes having other activities as well, such as, e.g., a lipase with phospholipase activity. The phospholipase activity may, e.g., be from a lipase with phospholipase side activity. In other embodiments of the invention the phospholipase enzyme activity is provided by an enzyme having essentially only phospholipase activity and wherein the phospholipase enzyme activity is not a side activity.

The phospholipase may be of any origin, e.g., of animal origin (such as, e.g., mammalian), e.g., from pancreas (e.g., bovine or porcine pancreas), or snake venom or bee venom. Preferably the phospholipase may be of microbial origin, e.g., from filamentous fungi, yeast or bacteria, such as the genus or species Aspergillus, e.g., A. niger; Dictyostelium, e.g., D. discoideum; Mucor, e.g., M. javanicus, M. mucedo, M. subtilissimus; Neurospora, e.g., N. crassa; Rhizomucor, e.g., R. pusillus; Rhizopus, e.g., R. arrhizus, R. japonicus, R. stolonifer; Sclerotinia, e.g., S. libertiana; Trichophyton, e.g., T. rubrum; Whetzelinia, e.g., W. sclerotiorum; Bacillus, e.g., B. megaterium, B. subtilis; Citrobacter, e.g., C. freundii; Enterobacter, e.g., E. aerogenes, E. cloacae; Edwardsiella, E. tarda; Erwinia, e.g., E. herbicola; Escherichia, e.g., E. coli; Klebsiella, e.g., K. pneumoniae; Proteus, e.g., P. vulgaris; Providencia, e.g., P. stuartdi; Salmonella, e.g., S. typhimurium; Serratia, e.g., S. liquefasciens, S. marcescens; Shigella, e.g., S. flexneri; Streptomyces, e.g., S. violeceoruber; Yersinia, e.g., Y. enterocolitica. Thus, the phospholipase may be fungal, e.g., from the class Pyrenomycetes, such as the genus Fusarium, such as a strain of F. culmorum, F. heterosporum, F. solani, or a strain of F. oxysporum. The phospholipase may also be from a filamentous fungus strain within the genus Aspergillus, such as a strain of Aspergillus awamori, Aspergillus foetidus, Aspergillus japonicus, Aspergillus niger or Aspergillus oryzae.

Preferred phospholipases are derived from a strain of Humicola, especially Humicola lanuginosa. The phospholipase may be a variant, such as one of the variants disclosed in WO 00/32758, which are hereby incorporated by reference. Preferred phospholipase variants include variants listed in Example 5 of WO 00/32758, which is hereby specifically incorporated by reference. In another preferred embodiment the phospholipase is one described in WO 04/111216, especially the variants listed in the table in Example 1.

In another preferred embodiment the phospholipase is derived from a strain of Fusarium, especially Fusarium oxysporum. The phospholipase may be the one concerned in WO 98/026057 displayed in SEQ ID NO: 2 derived from Fusarium oxysporum DSM 2672, or variants thereof.

In a preferred embodiment of the invention the phospholipase is a phospholipase A₁ (EC. 3.1.1.32). In another preferred embodiment of the invention the phospholipase is a phospholipase A₂ (EC.3.1.1.4.).

Examples of commercial phospholipases include LECITASE™ and LECITASE™ ULTRA, YIELSMAX, or LIPOPAN F (available from Novozymes A/S, Denmark).

Amylases: Suitable amylases (alpha and/or beta) include those of bacterial or fungal origin. Chemically modified or protein engineered mutants are included. Amylases include, for example, alpha-amylases obtained from Bacillus, e.g., a special strain of B. licheniformis, described in more detail in GB 1,296,839, or the Bacillus sp. strains disclosed in WO 95/026397 or WO 00/060060.

Examples of useful amylases are the variants described in WO 94/02597, WO 94/18314, WO 96/23873, WO 97/43424, WO 01/066712, WO 02/010355, WO 02/031124 and WO 2006/002643 (which references all incorporated by reference.

Commercially available amylases are DURAMYL™, TERMAMYL™, TERMAMYL ULTRA™, NATALASE™, STAINZYME™, STAINZYME ULTRA™, FUNGAMYL™ and BAN™ (Novozymes A/S), RAPIDASE™ and PURASTAR™ (from Genencor International Inc.).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Thielavia terrestris, Myceliophthora thermophila, and Fusarium oxysporum disclosed in U.S. Pat. No. 4,435,307, U.S. Pat. No. 5,648,263, U.S. Pat. No. 5,691,178, U.S. Pat. No. 5,776,757, WO 89/09259, WO 96/029397, and WO 98/012307.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO 1999/001544.

Commercially available cellulases include CELLUZYME™, CELLUCLAST™, CAREZYME™, ENDOLASE™, RENOZYME™ (Novozymes A/S), CLAZINASE™ and PURADAX HA™, ACCELERASE™ 1000 (Genencor International Inc.), and KAC-500(B)™ (Kao Corporation).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ and NovoZym™ 51004 (Novozymes A/S).

Pectate lyases (also called polygalacturonate lyases): Examples of pectate lyases include pectate lyases that have been cloned from different bacterial genera such as Erwinia, Pseudomonas, Klebsiella and Xanthomonas, as well as from Bacillus subtilis (Nasser et al., 1993, FEBS Letts. 335:319-326) and Bacillus sp. YA-14 (Kim et al., 1994, Biosci. Biotech. Biochem. 58: 947-949). Purification of pectate lyases with maximum activity in the pH range of 8-10 produced by Bacillus pumilus (Dave and Vaughn, 1971, J Bacteriol. 108: 166-174), B. polymyxa (Nagel and Vaughn, 1961, Arch. Biochem. Biophys. 93: 344-352), B. stearothermophilus (Karbassi and Vaughn, 1980, Can. J. Microbiol. 26: 377-384), Bacillus sp. (Hasegawa and Nagel, 1966, J. Food Sci. 31: 838-845) and Bacillus sp. RK9 (Kelly and Fogarty, 1978, Can. J. Microbiol. 24: 1164-1172) have also been described. Any of the above, as well as divalent cation-independent and/or thermostable pectate lyases, may be used in practicing the invention. In preferred embodiments, the pectate lyase comprises the amino acid sequence of a pectate lyase disclosed in Heffron et al., 1995, Mol. Plant-Microbe Interact. 8: 331-334 and Henrissat et al., 1995, Plant Physiol. 107: 963-976. Specifically contemplated pectate lyases are disclosed in WO 99/27083 and WO 99/27084. Other specifically contemplated pectate lyases derived from Bacillus licheniformis is disclosed as SEQ ID NO: 2 in U.S. Pat. No. 6,284,524 (which document is hereby incorporated by reference). Specifically contemplated pectate lyase variants are disclosed in WO 02/006442, especially the variants disclosed in the Examples in WO 02/006442 (which document is hereby incorporated by reference).

Examples of commercially available alkaline pectate lyases include BIOPREP™ and SCOURZYME™ L from Novozymes A/S, Denmark.

Mannanase: Examples of mannanases (EC 3.2.1.78) include mannanases of bacterial and fungal origin. In a specific embodiment the mannanase is derived from a strain of the filamentous fungus genus Aspergillus, preferably Aspergillus niger or Aspergillus aculeatus (WO 94/25576). WO 93/24622 discloses a mannanase isolated from Trichoderma reesei. Mannanases have also been isolated from several bacteria, including Bacillus organisms. For example, Talbot et al., 1990, Appl. Environ. Microbiol. 56(11): 3505-3510 describes a beta-mannanase derived from Bacillus stearothermophilus. Mendoza et al., 1994, World J. Microbiol. Biotech. 10(5): 551-555 describes a beta-mannanase derived from Bacillus subtilis. JP-A-03047076 discloses a beta-mannanase derived from Bacillus sp. JP-A-63056289 describes the production of an alkaline, thermostable beta-mannanase. JP-A-63036775 relates to the Bacillus microorganism FERM P-8856 which produces beta-mannanase and beta-mannosidase. JP-A-08051975 discloses alkaline beta-mannanases from alkalophilic Bacillus sp. AM-001. A purified mannanase from Bacillus amyloliquefaciens is disclosed in WO 97/11164. WO 91/18974 describes a hemicellulase such as a glucanase, xylanase or mannanase active. Contemplated are the alkaline family 5 and 26 mannanases derived from Bacillus agaradhaerens, Bacillus licheniformis, Bacillus halodurans, Bacillus clausii, Bacillus sp., and Humicola insolens disclosed in WO 99/64619. Especially contemplated are the Bacillus sp. mannanases concerned in the Examples in WO 99/64619 which document is hereby incorporated by reference.

Examples of commercially available mannanases include MANNAWAY™ available from Novozymes A/S Denmark.

### Liquid Deodorant Compositions

The present invention is also directed to a composition comprising Bacillus velezensis strain NRRL B-50150 in an aqueous solution. This composition is designed to provide short- and long-term odor control effects and is environmentally friendly and economical for use.

An operable concentration range for Bacillus velezensis strain NRRL B-50150 is from about 1×10⁵ CFU/ml to 1×10¹⁰ CFU/ml, e.g., from about 1×10⁶ CFU/ml to 1×10⁸ CFU/ml, with a preferred concentration being about 1×10⁸ CFU/ml, such as about 1×10⁷ CFU/ml of the formulation.

### Odor Neutralizer Components

The deodorant compositions of the present invention may further comprise an odor neutralizer, which is an agent that can rapidly interact, by chemical reactions, with odorous compounds to produce odorless compounds. These agents should not rely on the masking mechanism of a perfume to control odors. In addition, these agents must be safe for use and cost effective. Neutralizers must be compatible with the microbial components.

In one embodiment of the present invention, the neutralizer is propylene carbonate, which has the molecular formula C₄H₆O₃. A preferred product of propylene carbonate is available from commercial vendors such as Huntsman Chemical Corporation.

In combination with other components of the composition, propylene carbonate can effectively reduce odors, including amine and ammonia odors such as trimethylamine, dimethylamine, and ammonia, which are the major target odorous compounds. In addition, propylene carbonate does not inactivate the microbial components even after a long period of contact.

Other odor neutralizing compounds, such as sodium citrate, sodium bicarbonate, and sodium carbonate, may also be used in the formulation of this invention.

Preferably, the odor neutralizing is present in an amount of 1-15 wt. %, such as 2-10 wt. % of the composition.

### Other Microbial Components

Viable microorganisms, or mixtures thereof, which are capable of growing on and degrading common domestic, industrial, pet, and animal wastes, capable of surviving the formulations, and compatible with the formulations, and do not produce malodor while performing, may be used in the invention.

Other microorganisms which can be used in the compositions of the present invention include strains of Alcaligens, Bacillus, Enterobacter, Klebsiella, Lactobacillus, Nitrobacter, Nitrosomonas, Pseudomonas, and Streptococcus, which are known to produce enzymes which are capable of breaking down organic material which can cause odors on carpets or other fibrous materials.

### Other Ingredients

Other ingredients may be used in the deodorant compositions of the present invention, including surfactants, fragrances, and dyes.

Surfactants can wet and emulsify insoluble waste materials present in the treated system and inclusion of surfactants in the composition of the invention will add to it a cleaning capability. Furthermore, surfactants can be used to break down the insoluble wastes therefore increasing the availability of them to microbial degradation. Suitable surfactants for the invention include nonionic and anionic types. Preferably, the surfactant is present in an amount of 0-8 wt. %, such as 0-6 wt. % of the composition.

Fragrance and dye can be optionally added to mask the odor and to control the color of the composition of the invention, respectively, and for market appeal.

The fragrance and dye must be compatible with other ingredients of the composition.

### Drain Opener Formulations

The present invention is also directed to a drain opener formulation comprising Bacillus velezensis strain NRRL B-50150 in an aqueous medium.

The drain opener formulation may further comprise surfactant(s) and/or preservative(s). The product has numerous advantages over currently available drain openers; such as activity at pH's closer to neutral, and solubilizing ability for soaps, fats, oils and greases. It further provides for biological activity specific to carbohydrates, and establishes a biofilm in the drains and on downstream surfaces to continuously aid the natural biodegradative process.

The composition of the present invention comprises a stable suspension of viable microorganisms, surfactant(s), preservatives, and optional fragrances in an aqueous medium with a preferred pH of approximately 5 to 6.

An operable concentration range for the microorganisms is from about 1×10⁶ CFU/ml to 1×10⁹ CFU/ml, with a preferred concentration being about 1×10⁸ CFU/ml, such as about 1×10⁷ CFU/ml of the formulation.

Unlike typical detergents, which predominately clean only surfaces, the surfactant in the formulation of the present invention can solubilize grease and make it bioavailable. The surfactant can be any readily biodegradable surfactant, or a mixture of surfactants with low toxicity for the microorganisms contained within the system. The surfactant(s) should have a high grease solubilizing capability. Ionic surfactants or blends of nonionic/ionic surfactants having a hydrophile/lipophile balance approaching 10 are particularly preferred for the necessary grease solubilization. Typical surfactants suitable for use with the present invention include n-alkyl benzene sulfonates and alkyl sulfonates. Preferred nonionic surfactants include aliphatic alcohol alkoxylates, alcohol ethoxylates, polyalkylene oxide copolymers, alkyl phenol alkoxylates, carboxylic acid esters, carboxylic amides, and others. The surfactant is present in a concentration from about 3 to 10 weight percent.

The pH of the solution should be maintained as near as possible to neutral to insure adequate bacterial activity, and to minimize health risk, but be in a range compatible for surfactant activity and conducive to the survival of the bacteria. An operable pH range can be between about 3 to 10.

A preservative such as paraben, methyl paraben, or 1,2-benzisothiazolin-3-one is added to inhibit or prevent the growth of undesirable microbial contaminants in the product. The necessity for a preservative is greatest when the pH is near neutral, and the least when the pH is at the extreme ends of the operable range. The concentration of the preservative is determined by the vendor's recommendations. A typical concentration range for the preservative used in the example is from about 0.075 to 0.75 weight percent.

An additional optional preservative can be added specifically to preserve the spore form of the microorganisms. Methyl anthranilate in concentrations of from about 25 to 50 ppm (w/v) by weight has been found to be a satisfactory additive.

Optionally a chelating agent is added to enhance stabilization of the formulation.

A fragrance can optionally be added to mask the odor of the product components, and for market appeal. The fragrance must be compatible with the other components of the formulation.

### Sanitizer Formulations

The present invention also relates to sanitizer formulations comprising Bacillus velezensis strain NRRL B-50150. The formulations comprise a suspension of a sanitizing composition, bacterial spores, and surfactants all contained in an aqueous solution. These formulations have the advantages of being a good surface cleaning agent and a good sanitizer along with providing the long term effect of beneficial bacteria that control pathogens and degrade wastes both on the surface and in the sewage system receiving the surface rinsate.

Sanitizing agents or composition and disinfectants belong to the same category of antimicrobial (active) ingredient. Antimicrobial (active) ingredients are compounds that kill microorganisms or prevent or inhibit their growth and reproduction and that contribute to the claimed effect of the product in which it is included. More specifically, a sanitizer is an agent that reduces the number of microbial contaminants or pathogens to safe levels as judged by public health requirements.

The surfactant component functions to clean the surface by removing the soil, dirt, dried urine and soap and helps in sanitizing the surface. The sanitizing composition sanitizes the surface (kills pathogens) and preserves the formulation from contamination by unwanted microorganisms. The bacterial spores and vegetative cells function to seed the waste collection system, control odor and provide a healthy dominant microbial population that inhibits the growth of pathogens through substrate competition, production of antibiotics, etc.

In one embodiment of the present invention, the composition comprises 1,2-benzisothiazolin-3-one (Proxel), tetrasodium ethylenediaminetetraacetate (EDTA), and isopropyl alcohol (IPA) at a selected range of concentrations, combined with other components of the formula, can effectively inactivate indicator organisms. This sanitizing composition preferably is at neutral pH and does not contain chlorine-related materials, which are commonly used as sanitizers. Consequently, this sanitizing composition is more environmentally friendly and less or not corrosive.

When the formulation is applied to a bathroom fixture, sink, toilet bowl, etc., it can be sprayed or squeezed out of a container directly onto a surface or brush. The formulation is then left on the surface or scoured against the surface with a brush for not less than 10 minutes. The product is then flushed or rinsed with water and discharged from the fixture.

The formulations of the invention contain sanitizing agents, bacterial spores, and surfactants. Fragrance and dye are also added to control smell and color of the formulations, respectively. Depending on the intended use, the formulation can optionally contain an abrasive. While the key components remain the same, different thickening agents might be used in the formulation with and without an abrasive.

Although many sanitizing agents can be used for inactivating pathogens on surfaces, not all of them can be used in the present invention. This is because the sanitizing agents used in this invention are not only required to inactivate pathogens effectively, but must not have negative effects on the stability and activity of the bacterial spores contained in the formulation. In addition, the sanitizing agents are required to be relatively friendly to the environment, and should not cause skin sensitization, and should not corrode the construction materials of the fixtures on which they are used.

In an embodiment, the sanitizing composition is composed of Proxel, EDTA, and IPA at selected ranges of concentrations. The maximum concentration of Proxel not likely to cause skin sensitization is about 2,900 mg/L. The suitable concentration ranges of Proxel, Versene (Versene contains 39% EDTA), and IPA are 0.087 to 0.29% (vol.), 0.36 to 1.19% (vol.), and 3.5 to 7% (vol.), respectively. An additional compound, methyl anthranilate, may also be used in the formulations of the invention. The purpose of using methyl anthranilate is to assist in preservation of the formulations.

Other sanitizing agents, such as quaternary ammonium compounds (QACs), nitro-containing organosulfur and sulfur-nitrogen compounds, may also be used in the formulation of this invention.

An operable concentration range for the microorganisms is from 1×10⁵ to 1×10⁹ CFU/ml, such as 10⁷ CFU/ml (CFU, colony forming unit) of the formulation.

### Surfactants

Surfactants are also an essential component in the sanitizer formulations of the present invention. The surfactants can wet and emulsify soil, including dirt, dried urine, soap, etc., present on a dirty surface. In addition, surfactants aid in the sanitization of the surface. Unlike surfactants usually used for surface cleaning, the surfactants used in the present invention have low toxicity for the microorganisms contained within the formulation. A single surfactant or a blend of several surfactants can be used.

Nonionic surfactants are generally preferred for use in the compositions of the present invention since they provide the desired wetting and emulsification actions and do not significantly inhibit spore stability and activity. Nonionic surfactants are surfactants having no electrical charge when dissolved or dispersed in an aqueous medium. Preferred nonionic surfactants include aliphatic alcohol alkoxylates, alcohol ethoxylates, polyalkylene oxide copolymers, alkyl phenol alkoxylates, carboxylic acid esters, carboxylic amides, and others.

Anionic surfactants or mixtures of anionic and nonionic surfactants may also be used in the formulations of the invention. Anionic surfactants are surfactants having a hydrophilic moiety in an anionic or negatively charged state in aqueous solution. Commonly available anionic surfactants include sulfonic acids, sulfuric acid esters, carboxylic acids, and salts thereof.

### Abrasives, Thickening Agents, Fragrance, and Dyes

Abrasives are water-insoluble solid particles. The purpose of using abrasives is to provide deep scouring and cleaning. Depending on the application, abrasives may be optionally used in the formulation of the invention. Suitable abrasives include calcium carbonate, magnesium carbonate, silica, etc. The preferred particle size of the abrasive ranges from about 90 to 325 mesh.

Since the specific gravity of bacterial spores is usually higher than that of water, a thickening agent needs to be used in this invention to suspend the spores. Suitable aqueous thickening agents include: polyacrylic acid, polystyrene, polyvinyl alcohol, polypropylene, etc. A preferred thickening agent for suspending bacterial spores is polyacrylic acid (e.g., Acrysol TT615 from Rohm and Haas Co.). If an abrasive is used in the formulation, thickening agents in addition to polyacrylic acid might be needed to maintain the suspension of the abrasive.

A fragrance and a dye can be optionally added to mask the odor and to control the color of the product components, respectively, and for market appeal. The fragrance and dye must be compatible with the other components of the formulation.

### Deposit of Biological Material

A Bacillus velezensis strain was deposited under the terms of the Budapest Treaty on Jun. 25, 2008 with the Agricultural Research Service Culture Collection, 1815 North University Street, Peoria, Ill. 61604, U.S.A., under accession number NRRL B-50150. The deposit shall be maintained in viable condition at the depository during the entire term of the issued patent and shall be made available to any person or entity for non-commercial use without restriction, but in accordance with the provisions of the law governing the deposit.

The following examples are given as exemplary of the invention but without intending to limit the same.

### EXAMPLES

### Materials & Methods

### Media and Reagents:

Chemicals used as buffers and reagents were commercial products of at least reagent grade.

### Plate Count Broth (cat. #275120, Difco-Becton Dickinson, Sparks, Md.) (“PCB”)

Standard Methods agar plates (SMA plates) (Smith River Biologicals, Ferrum, Va. cat. #11-00450)

### Marine Agar 2216 (cat. #212185, Difco-Becton Dickinson, Sparks, Md.)

### Marine Broth 2216:(cat. #279110, Difco-Becton Dickinson, Sparks, Md.)

### Bacto-Peptone (cat. #211677, Difco-Becton Dickinson, Sparks, Md.)

### Yeast Extract (LD) (cat. #210933, Difco-Becton Dickinson, Sparks, Md.)

Soluble Starch (cat. #S-2630, Sigma, St. Louis, Mo.)
R1 and R2 buffers (cat. #11876473 316; Roche, Indianapolis, Ind.)

### Equipment

### Konelab Arena 30 (Thermo Electron Corporation, Vantaa, Finland)

### BioTek Synergy Kinetic Plate Reader (Winooski, Vt.)

### Example 1

### Enzyme Production Procedure:

Enzyme production medium is used according to the following recipe:

Base Media (all values in q/L unless otherwise noted)

| | |
|---|---|
| Bacto-Peptone | 5 |
| NaCl | 2.5 |
| Yeast Extract | 3 |
| Soluble Starch | 1 |

Materials are mixed into diH₂O and autoclaved 20 min.

10 ml overnight cultures of strains are grown in PCB at 35° C. with shaking at 200 rpm. The next day, 0.2 ml of this culture is used to inoculate 100 ml of enzyme production medium. This culture is grown at 35° C. with shaking at 200 rpm. All culture flasks are grown for 80 hours at 35° C. with shaking at 200 rpm.

Over the course of 80 hours at 8-12 hour frequencies, 3 ml of culture is removed, centrifuged, filtered and 2 ml of the filtrate is added to a plastic tube containing 1.0 ml of sterile 50% glycerol. The tube is labeled and stored at −20° C. until all samples are ready for analysis. Amylase assay:

Alpha-amylases (1,4-α-D-glucanohydrolases, E.C. 3.2.1.1) catalyze the hydrolytic degradation of polymeric carbohydrates such as amylose, amylopectin and glycogen by cleaving 1,4-alpha-glucosidic bonds. In polysaccharides and oligosaccharides, several glycosidic bonds are hydrolyzed simultaneously. Maltotriose, the smallest such unit, is converted into maltose and glucose, albeit very slowly. The kinetic method described here is based on the well-proven cleavage of 4,6-ethylidene-(G7)-1,4-nitrophenyl-(G1)-α,D-maltoheptaoside by alpha-amylase and subsequent hydrolysis of all the degradation products to p-nitrophenol with the aid of alpha-glucosidase. This results in 100% liberation of the chromophore.

This process has been automated in the Konelab Arena 30 with the following steps:

1) 200 microliters of R1 reagent is pipetted into cuvette,
2) 16 microliters of sample is added to cuvette,
3) Mixture is incubated for 300 seconds to obtain temperature of 37° C.,
4) 20 microliters of R2 reagent is pipetted into cuvette and mixture is incubated for 180 seconds, and
5) Absorption is measured every 18 seconds at 405 nm for a total of 7 measurements for each sample.

Defined oligosaccharides are cleaved under the catalytic action of alpha-amylases. The resulting PNP derivatives are cleaved directly to PNP by the action of alpha-glucosidase and the color intensity of the p-nitrophenol formed is directly proportional to the alpha-amylase activity and is measured spectrophotometrically.

5 ethylidene-G₇PNP+H₂O→2 ethylidene-G₅+2 G₂PNP+2 ethylidene-G₄+2 G₃PNP+ethylidene-G₃+G₄PNP (1)

2 G₂PNP+2 G₃PNP+G₄PNP+14H₂O→5 PNP+14G (2)

Reaction (1) is mediated by the amylase added from the standard or sample. Reaction (2) is mediated by the alpha-glucosidase provided in the kit.

### Unit Definition

BAN is an alpha-amylase available from Novozymes. The analytical standard was supplied at 360 KNU(B)/g=360 NU(B)/mg.

### Specificity and Sensitivity

Because each amylase will have a different specificity, the samples should be diluted such that the final slopes read from the Konelab are between 0.05 and 0.50 to make sure that the experimental samples fall within the scope of the standard curve.

Bacillus velezensis strain NRRL B-50150 produced amylase activity in these assays.

### Example 2

### Phage Sensitivity Assay

Bacillus velezensis strain NRRL B-50150 and Bacillus velezensis strain SB3190 were grown in buffered plate count broth (BPCB: 17 g m-Plate Count Broth Difco, 20 ml of pH 7 buffer made with 1 part 9.078 g/L KH₂PO₄ and 1.5 parts 9.476 g/L of K₂HPO₄, pH adjusted to 7) to a density of approximately 0.2 absorbance units at 590 nm wavelength. 100 microliters of each culture were delivered to wells of a 96 well BD Oxygen Biosensor microtiter plate (Catalog #353830, BD Lifesciences, San Jose, Calif.). The cultures were diluted in additional BPCB and a 0.01× dilution of the cultures was delivered to additional wells of the same plate. Each dilution of bacterial culture received 100 microliters of five different concentrations of phage challenge as follows: 1× (˜10¹⁰ pfu/ml), 0.1×, 0.01×, 0.001×, and 0.0001×. The diluent for the phage was BPCB. One well of each bacterial culture dilution received 100 microliters of plain BPCB instead of phage and thus served as the control well. Plates were read on a BioTek Synergy kinetic plate reader at 485/20 nm excitation, 645/40 nm emission at 20 minute intervals for 20+hours with 10 seconds of mixing at level 4 before each read. The BD Oxygen Biosensor microtiter plates contain an oxygen sensitive fluorophore that fluoresces when the cell culture in the well consumes oxygen and thus fluorescence intensity correlates to culture growth rates and general health. Data was analyzed by comparing the fluorescent O₂ consumption curves of Bacillus velezensis strain NRRL B-50150 to Bacillus velezensis strain SB3190 at the various bacteria and phage ratios. Increasing fluorescence (bacterial growth) without decreases or plateaus (lysis or decreased growth rate) in the presence of phage was interpreted as resistance to phage. Bacillus velezensis strain NRRL B-50150 outperformed Bacillus velezensis strain SB3190 in this way at multiple cell and phage densities examined. At 1× and 0.1× cell culture concentration, Bacillus velezensis strain SB3190 succumbed to phage pressure at most phage concentrations tested by showing a noticeable depression in O2 consumption, whereas Bacillus velezensis strain NRRL B-50150 showed ample, unwavering, and prolonged proliferation at all phage concentrations.

### Example 3

Petri Plate V. harveyi Zone of Inhibition

Bacillus velezensis strain NRRL B-50150 and V. harveyi (ATCC 25919) were grown separately in plate count broth and marine broth, respectively, for 18 to 20 hours at 28° C. with shaking. V. harveyi culture was swabbed to form a lawn on the surface of Marine Agar (Difco) and a 5 mm hole was bored into the agar with a sterile stainless steel tube. 50 microL of Bacillus velezensis strain NRRL B-50150 liquid culture was delivered into the hole in the agar and the plate was incubated for 24 hours at 30° C., agar side down. Inhibited V. harveyi lawn in proximity to the hole was scored as positive biocontrol for Bacillus velezensis strain NRRL B-50150. The diameter of the zone of inhibition (including the hole) was measured in millimeters (mm) to allow semi-quantitative assessment of control. Bacillus velezensis strain NRRL B-50150 zone diameter was 11 mm.

### Example 4

Petri Plate E. coli Zone of Inhibition

Bacillus velezensis strain NRRL B-50150 and E. coli (ATCC 43827) were grown separately in plate count broth for 18 to 20 hours at 28° C. with shaking. E. coli culture was swabbed to form a lawn on the surface of Standard Methods Agar and a 5 mm hole was bored into the agar with a sterile stainless steel tube. 50 microL of Bacillus velezensis strain NRRL B-50150 liquid culture was delivered into the hole in the agar and the plate was incubated for 24 hours at 30° C., agar side down. Inhibited E. coli lawn in proximity to the hole was scored as positive biocontrol for Bacillus velezensis strain NRRL B-50150. The diameter of the zone of inhibition (including the hole) was measured in millimeters (mm) to allow semi-quantitative assessment of control. Bacillus velezensis strain NRRL B-50150 zone diameter was 15 mm.

### Example 5

Petri Plate Salmonella typhimurium Zone of Inhibition

Bacillus velezensis strain NRRL B-50150 and Salmonella typhimurium (Novozymes Culture Collection) were grown separately in plate count broth for 18 to 20 hours at 28° C. with shaking. Salmonella typhimurium culture was swabbed to form a lawn on the surface of Standard Methods Agar and a 5 mm hole was bored into the agar with a sterile stainless steel tube. 50 microL of Bacillus velezensis strain NRRL B-50150 liquid culture was delivered into the hole in the agar and the plate was incubated for 24 hours at 30° C., agar side down. Inhibited Salmonella typhimurium lawn in proximity to the hole was scored as positive biocontrol for Bacillus velezensis strain NRRL B-50150. The diameter of the zone of inhibition (including the hole) was measured in millimeters (mm) to allow semi-quantitative assessment of control. Bacillus velezensis strain NRRL B-50150 zone diameter was 10 mm.

### Example 6

Petri Plate Pseudomonas aeruainosa Zone of Inhibition

Bacillus velezensis strain NRRL B-50150 and Pseudomonas aeruginosa Pa-01 (gift from Montana State University, lab of Ann Camper) were grown separately in plate count broth for 18 to 20 hours at 28° C. with shaking. Pseudomonas aeruginosa culture was swabbed to form a lawn on the surface of Standard Methods Agar and a 5 mm hole was bored into the agar with a sterile stainless steel tube. 50 microL of Bacillus velezensis strain NRRL B-50150 liquid culture was delivered into the hole in the agar and the plate was incubated for 24 hours at 30° C., agar side down. Inhibited Pseudomonas aeruginosa lawn in proximity to the hole was scored as positive biocontrol for Bacillus velezensis strain NRRL B-50150. The diameter of the zone of inhibition (including the hole) was measured in millimeters (mm) to allow semi-quantitative assessment of control. Bacillus velezensis strain NRRL B-50150 zone diameter was 11 mm.

While specific embodiments of the invention have been illustrated and described herein, it is realized that modifications and changes will occur to those skilled in the art. It is therefore to be understood that the appended claims are intended to cover all modifications and changes as fall within the true spirit and scope of the invention.

1. A method for preventing and/or reducing biofilm formation on a surface, comprising subjecting said surface to Bacillus velezensis strain NRRL B-50150.

2. The method of claim 1, wherein the surface is a hard surface, preferably made of one or more materials selected from the group consisting of metal, plastics, rubber, board, glass, wood, paper, concrete, rock, marble, gypsum, and ceramic materials, such as porcelain; or a soft surface, preferably made of one or more materials selected from the group consisting of fibers, e.g., yarns, textiles, vegetable fibers; rock wool, hair; skin; keratinous materials; and internal organs, e.g., lungs; or a porous surface.

3. The method of claim 1, wherein the hard surface is a toilet bowl; toilet water reservoir; cooling tower; water treatment plant; water tank; dairy, food processing plant; chemical or pharmaceutical process plant; or medical device.

4. The method of claim 1, wherein the biofilm formation is caused by one or more undesired microorganisms, preferably bacteria, such as pathogenic bacteria.

5. The method of claim 4, wherein the undesired microorganism, preferably bacteria, causes corrosion, pitting, degradation of the material in question; infection; staining or otherwise making a surface appear aesthetically unpleasing.

6. The method of claim 1, wherein the method is repeated periodically.

7. The method of claim 1, further comprising subjecting the surface to an enzyme, preferably an enzyme selected from the group of proteases, alpha-amylases, cellulases, lipases, peroxidases/oxidases, pectate lyases, and mannanases, or mixtures thereof.

8. The method of claim 1, further comprising subjecting the surface to one or more agents selected from the group consisting of dispersants, surfactants, anti-microbial agents, and biocides.

9-13. (canceled)

14. A composition comprising Bacillus velezensis strain NRRL B-50150.

15. The composition of claim 14, which further comprises a surfactant.

16. The composition of claim 14, which further comprises one or more enzymes.

17. The composition of claim 16, wherein the enzyme is selected from the group consisting of proteases, alpha-amylases, cellulases, lipases, peroxidases/oxidases, pectate lyases, and mannanases, or mixtures thereof.

18. The composition of claim 14, which further comprises one or more ingredients selected from the group consisting of dispersants, stabilizers, anti-microbial agents, fragrances, dyes, and biocides.

19. The composition of claim 14 which is a liquid deodorizing composition.

20. The composition of claim 14, wherein Bacillus velezensis strain NRRL B-50150 is present in a concentration of from about 1×10⁵ to 1×10¹⁰ per ml.

21. The composition of claim 14, further comprising an odor neutralizing component which functions to provide for rapid odor reduction.

22. The composition of claim 21, wherein the odor neutralizing component comprises propylene carbonate.

23. The composition of claim 21, wherein the odor neutralizing component is at least one selected from the group consisting of sodium citrate, sodium bicarbonate, and sodium carbonate.

24. The composition of claim 14, further comprising one or more microbes selected from the group consisting of Alcaligens, Bacillus, Enterobacter, Klebsiella, Lactobacillus, Nitrobacter, Nitrosomonas, Pseudomonas, and Streptococcus.

25. A biologically pure culture of Bacillus velezensis strain NRRL B-50150.
